(19) United States
(12) Patent Application Publication
(10) Pub. No.: US 2017/0340774 A1
(43) Pub. Date: Nov. 30, 2017

(54) POLETHYLENE GLYCOL HYDROGEL INJECTION

(71) Applicant: SUNBIO INC., Gyeonggi-do (KR)

(72) Inventors: Kwang NHO, Anyang-si (KR); Min Jung AHN, Ansan-si (KR); Byung Hee SOHN, Seoul (KR)

(21) Appl. No.: 15/619,947

(22) Filed: Jun. 12, 2017

Related U.S. Application Data
(63) Continuation-in-part of application No. PCT/KR2016/006434, filed on Jun. 17, 2016.

(30) Foreign Application Priority Data
Sep. 30, 2015 (KR) 10-2015-0138210

(51) Int. Cl. A61L 27/26; A61L 27/52

(52) U.S. Cl. CPC A61L 27/26; A61L 27/52; A61L 2400/06

(57) ABSTRACT

The present invention relates to a polyethylene glycol hydrogel injection, and, more particularly, to an injection to be administered into a joint (a synovial joint cavity) for the improvement of symptoms of arthritis by containing two separate buffer solutions, wherein a solution (1) contains a polyethylene glycol derivative with an electrophilic functional group and a buffer of pH 3.5 to 6, and a solution (2) contains a polyethylene glycol derivative with a nucleophilic functional group, hyaluronic acid, and a buffer of pH 7.5 to 11. The injection of the present invention is highly biocompatible and long-lasting in the joint, showing the efficacy of pain relief, cartilage protection, and inhibition of inflammation, thus offering the effective prevention and treatment of arthritis.

### RELATED APPLICATION

This application is a Continuation-in-Part (CIP) of PCT Patent Application No. PCT/KR2016/006434 having International filing date of Jun. 17, 2016, which claims the benefit of priority of Korean Patent Application No. 10-2015-0138210 filed on Sep. 30, 2015.

The contents of the above applications are all incorporated by reference as if fully set forth herein in their entirety.

### FIELD AND BACKGROUND OF THE INVENTION

The present invention relates to a polyethylene glycol hydrogel injection.

Osteoarthritis is a joint disease that is characterized by severe pain due to synovial inflammation and bone exposure due to the loss of articular cartilage around subchondral bone, and is caused by the structural deformation and degeneration of a joint. It mainly affects the joints that carry weight, thus resulting in severe pain, restriction of daily activities, and structural deformities. It has been suggested that osteoarthritis may be caused by genetics, injuries to the joints, repetitive usage of particular joints, or obesity. Osteoarthritis is especially common in the elderly population, and the rate of the disease has been escalating due to increased lifespan. Thus far, treatments of osteoarthritis have been focused on relieving pain by ameliorating symptoms, delaying the progression of the disease to attenuate the deterioration of the joints, and ultimately increasing the quality of life, rather than rectifying a structural damage of the joint. Injection into a synovial joint cavity is one of the treatment options for osteoarthritis; a steroid or hyaluronic acid injection is the most common treatment. Steroid injections have shown to temporarily relieve 80 to 90% of pain, though frequent injections of steroids have been reported to severely damage the joints, thus it is recommended that the injection to be performed at 4 to 6-month intervals. Hyaluronic acid injections are designed to supplement, hyaluronic acid, which is deficient due to inflammation by osteoarthritis, from an external source. Hence, the injection of hyaluronic acid is also called ‘viscosupplementation’.

Hyaluronic acid viscosupplementation originated in the 1970s, with the development of products such as Healon® and Hylartil-Vet®, when it was being used in veterinary medicine for race horses. In 1987, the Seikagaku Corporation and Fidia Farmaceutici s.p.a. developed Artz® and Hyalgan® respectively, as treatments for human osteoarthritis. Since then, Synvisc® was developed by Balazs et al., in the 1990s as a result of continued research and development for treatment using hyaluronic acid. Nowadays, there are numerous single injection products of low-molecular-weight hyaluronic acid in various compositions being sold, which have been used for supplemental therapy in Japan and they are being extensively used worldwide including Korea and Europe (Advancing Viscosupplementation. 2007. Dr. Ting Choon Meng).

Early phase hyaluronic acid production involved in vivo extraction, but recently, hyaluronic acid is being mass produced by a microbial fermentation process. In vivo extraction of hyaluronic acid is typically performed on a rooster comb, which contains about 1% hyaluronic acid. The hyaluronic acid from a rooster comb has average molecular weight of 10 million daltons (Da). The hyaluronic acid may shift to a lower molecular weight during extraction and purification, and about 5 million Da of hyaluronic acid may be obtained in the end. The hyaluronic acid produced by microbial fermentation may be produced using Streptococcus zooepidemicus or Streptococcus epui bacteria. The hyaluronic acid of these strains is almost identical to the hyaluronic acid of living tissue in terms of the structure and characteristics, which may be useful for mass production. There is a recent increase in hyaluronic acid demand, thus mass production using the microbial fermentation process is also increasing every year [“Function and application of hyaluronic acid”
┌Food & Packaging (Japan), 54(3), 2013, 138-142].

Hyaluronic acid, in which β-D-N-acetylglucosamine and β-D-glucuronic acid are alternately linked to form a large linear complex carbohydrate, has high average molecular weight and naturally resides in extracellular matrices. Since hyaluronic acid has excellent biocompatibility and viscoelasticity, it is being widely used for medical and cosmetic purposes. When hyaluronic acid is injected into a synovial joint cavity, it can relieve pain and improve the joint condition by lubricating the joint area and absorbing shock. To investigate therapeutic effects of hyaluronic acid, Euflexxa® (1% hyaluronic acid) was injected three times once a week for 6 months, after removing synovial fluids from 14 adults and 14 elderly people who suffer from osteoarthritis. As a result, it was reported that there was average 51.2% reduction in pain at a walking point [The Open Orthopedics Journal, 2013, 7, 378-384]. The molecular weight of hyaluronic acid from synovial fluid in a healthy adult is 6,000,000 Da. When the pharmacokinetic values were analyzed at 2.5 Hz, viscosity was 45 Pa, and elasticity was 117 Pa (Disorders of the knee 2nd ed. J B Lippincott; 1982). Commercially available hyaluronic acid products at present are categorized into either the products consisting of linear hyaluronic acid itself or the products consisting a cross-linked gel of hyaluronic acid. Sodium hyaluronate solution products, such as Hyalgan®, ARTZ®, Euflexxa®, and ORTHOVISC®, contain hyaluronic acid with molecular weights between 500,000 to 3,600,000 Da. When 2 ml of these products are repeatedly injected 3 times or 5 times, they have shown to relieve pain due to osteoarthritis for 3 months or 6 months, respectively. Products in the form of crosslinked hyaluronic acid, such as Synvisc®, Synvisc-One®, Durolane®, Gel-One®, and MONOVISC® that was FDA-approved most recently aim at increasing the hyaluronic acid molecular weight by crosslinking or increasing the sustainability of therapeutic effects of these products by protecting a site in hyaluronic acid that is susceptible to degradation by other enzymes.

U.S. Pat. No. 4,582,865 discloses that hyaluronic acid (HA) and divinyl sulfone (DVS) in a basic buffer solution readily react to form a cross-linked HA gel, and by varying the reaction conditions (polymer/DVS ratio, molecular weight and concentration of hyaluronic acid, etc.,) which can be conveniently used to control the swelling ratio of the cross-linked HA gel. The crosslinking conditions include a hyaluronic acid molecular weight between 50,000 to 8,000,000 Da, with a concentration between 1 to 8%. The HA/DVS weight ratio can be from 15:1 to 1:5 and lower. This reaction is usually carried out in pH 9.0, at room temperature, i.e., about 20° C. At higher reaction temperatures than 20° C., HA can degrade relatively rapidly in alkaline solutions. at elevated temperatures and, If such degradation occurs, the decrease in MW can affect the properties of the obtained gels. Synvisc® and Synvisc-One® are the examples of commercially available injection-types of cross-linked HA gel using the method, and disclosed are cross-linked gels of hyaluronic acid, alone or mixed with other hydrophilic polymers and containing various substances or covalently bonded low molecular weight substances and processes for preparing them.

In addition, U.S. Pat. No. 5,827,937 discloses methods of forming a biocompatible polysaccharide gel, in particular, utilizing 1,4-butanedioldiglycidylether (BDDE), which contains an epoxy functional group as a polyfunctional crosslinking agent with hyaluronic acid to form an elastic hydrogel. 0.2% crosslinking agent and 10% hyaluronic acid at pH 9 were used to form an ether bond primarily by a crosslinking reaction. Then, by adding acetic acid, pH is lowered to 2 to 6 to cause a secondary reaction of forming an ester bond for gelation. Increased gelation density or concentration with biologically active substances (hormone, cytokine, vaccine, cell, etc.) was designed for sustained release or better preservation of a hydrogel and such a biocompatible composition of a hydrogel may be administered for medical or prophylactic purposes. Durolane® is an example of a commercially available hyaluronic acid product, which uses the method described above and culturing of microorganisms (Streptococcus equi.) to generate high molecular weight (9,000,000 Da) hyaluronic acid with high purity.

The references illustrate methods and compositions of forming crosslinked hydrogel by allowing a hydroxyl group of hyaluronic acid and crosslinking agents (DVS, BDDE) to bind to each other. On the other hand, U.S. Pat. No. 6,031,017 discloses methods of hyaluronic acid hydrogel formation by first generating photoreactive hyaluronic acid derivative using cinnamic acid, and then producing hydrogel to form a cyclobutane ring by UV. In a 10 Hz condition, dynamic viscoelasticity values measured by a rheometer indicate a storage modulus (G′: elasticity) is 50˜1500 Pa and a loss modulus (G″: viscosity) is 10˜300 Pa, which suggest superior viscoelasticity compared to the others. Conventional crosslinking agents may result in insoluble form, and difficulty in separating and removing low molecular weight compounds and toxic crosslinking agents from the crosslinked structure. However, photoreactive hyaluronic acid derivatives do not directly form tertiary structures, thus facilitate removal of the unreacted low molecular weight compounds. In addition, they are water soluble, which give the photoreactive hyaluronic acid derivatives an enormous advantage. GEL-ONE®, which is produced using the aforementioned method, has more persistent effects compared to the other products due to amination of the carboxyl group in hyaluronic acid, thus results in attenuated degradation of hyaluronic acid. Because hyaluronic acid has a relatively short half-life after being administered into the body, there have been rigorous efforts to extend the half-life and the efficacy of hyaluronic acid by increasing the gelation composition and the hyaluronic concentration. However, doing so would also increase the viscosity of the hydrogel, which may lead to an increase in injection force during administration. An increased injection force may not only be technically challenging during administration to patients, but also may present a physical burden to both patients and healthcare providers. Despite crosslinked hyaluronic acid displays extended half-life compared to non-crosslinked hyaluronic acid, the crosslinked hyaluronic acid still has a low sustainability in a human body as it is degraded within 6-month upon administration.

### SUMMARY OF THE INVENTION

### Technical Problem

Hence, in order to reduce a risk of causing pain due to the high injection force during administration into a joint (a synovial joint cavity), which existing products have and results from the high viscosity of the products, the present inventors developed an injection formulation that effectively relieves pain caused by arthritis, protects cartilage, and suppresses synovial inflammation even with single injection into a joint, and thus completed the present invention. Such an injection exhibits high biocompatibility and sustainability in a human body by being easily injectable with a syringe due to low viscosity at the time of injection and causing a reaction between two types of polyethylene glycol (PEG) derivatives to gradually form a peptide bond, which leads to the formation of hydrogel containing hyaluronic acid and exhibiting excellent viscoelasticity. Here, the low viscosity at the time of injection is achieved by controlling the duration of crosslinking.

Therefore, the present invention is directed to providing a PEG hydrogel injection, which is a hydrogel using a PEG derivative including hyaluronic acid. The injection also has high biocompatibility, because the viscosity thereof is low at the time of injection into a joint for the ease of injection and increases in the joint after administration

### Technical Solution

To fulfill the objectives, the present invention provides an injection containing two separate buffer solutions, wherein a solution (1) contains a PEG derivative with electrophilic functional group and a buffer of pH 3.5 to 6, and a solution (2) contains a PEG derivative with nucleophilic functional group, hyaluronic acid, and a buffer of pH 7.5 to 11.

Again to fulfill the aforementioned objectives, the present invention provides a kit for the injection, wherein the kit includes a buffer solution set (1) containing a PEG derivative powder with electrophilic functional group and a buffer of pH 3.5 to 6; and a buffer solution set (2) containing a PEG derivative powder with nucleophilic functional group and a buffer of pH 7.5 to 11 containing hyaluronic acid, wherein the buffer solution set (1) and the buffer solution set (2) are stored in separate containers.

### Advantageous Effects

The composition of the present invention is intended for administration into a joint (a synovial joint cavity) aimed at improving and treating various conditions of osteoarthritis, which, in sync with an aging society, commonly occurs in the elderly population. Such a composition exhibits enhanced pain relief, cartilage protection, and synovial membrane inflammation inhibition that are sustained with single injection, without requiring surgery, and can be used as an injection composition with excellent biocompatibility and ease of administration targeting the interior of joints.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIG. 1 is a schematic diagram of the formation of a hydrogel by injecting an injection of the present invention.

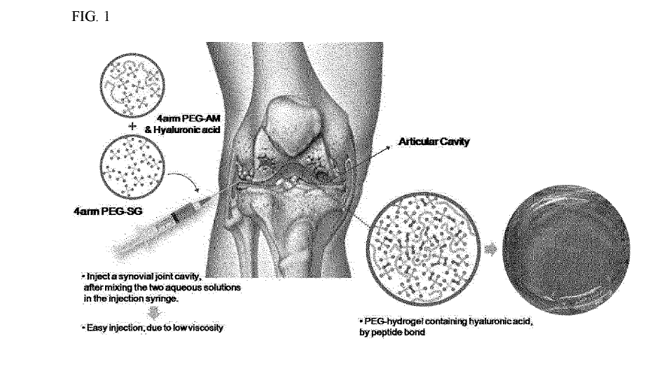

FIG. 2 is a graph representing the complex viscosity values of a hydrogel that is formed after the injection of an injection of the present invention.

FIG. 3 is a graph representing the effect of joint pain relief of an injection of the present invention.

FIG. 4 is a graph representing the radioactivity recovered following dosing from the knees, over time [Ave. % Recovery of [¹⁴C] example 2 in Rabbit Knees].

### DESCRIPTION OF SPECIFIC EMBODIMENTS OF THE INVENTION

Hereinafter, exemplary embodiments of the present invention will be described in detail. However, the present invention is not limited to the embodiments disclosed below and can be implemented in various forms. The following embodiments are described in order to enable those of ordinary skill in the art to embody and practice the present invention.

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of exemplary embodiments. The term “and/or” includes any and all combinations of one or more of the associated listed items.

The terminology used herein is for the purpose of describing particular embodiments only and is not intended to be limiting the exemplary embodiments. The singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, components and/or groups thereof and do not preclude the presence or addition of one or more other features, integers, steps, operations, elements, components and/or groups thereof.

With reference to the appended drawings, exemplary embodiments of the present invention will be described in detail below. To aid in understanding of the present invention, like numbers refer to like elements throughout the description of the figures, and the description of the same elements will not be iterated.

The present invention relates to an injection containing a first buffer solution (solution 1) containing a polyethylene glycol (PEG) derivative with electrophilic functional group and a buffer of pH 3.5 to 6; and a second buffer solution (solution 2) containing a PEG derivative with nucleophilic functional group, hyaluronic acid, and a buffer of pH 7.5 to 11.

The PEG derivative with electrophilic functional group may be a compound represented by the following Structural Formula 1A:

Core-[—(CH₂CH₂O)ₙ—(CH₂)ₘ₁-(L)ₚ-(CH₂)ₘ₂—R]_{q} [Structural Formula 1A]

where in formula 1A, L is a linker, may be each independently selected from the group consisting of

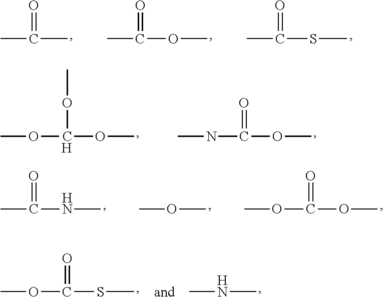

R is a functional group selected from the group consisting of

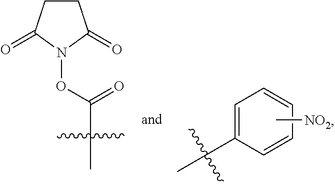

which may react with an amine group to form a peptide bond,

Core is selected from the group consisting of

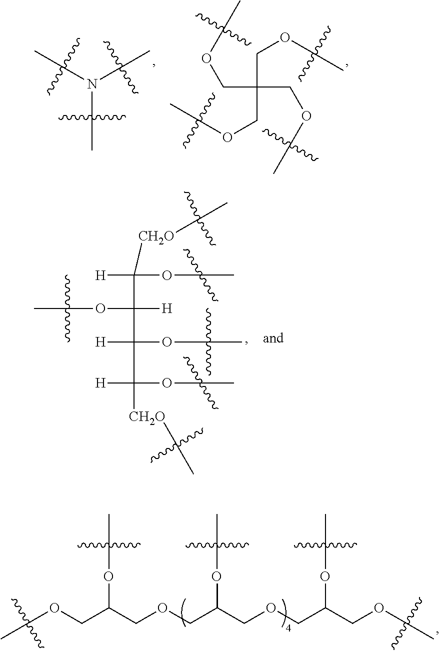

n is an integer from 10 to 2000,

m₁ and m₂ are each independent integers from 0 to 3,

p is an integer from 0 to 1,

q is an integer from 3 to 8.

An exemplary PEG derivative with an electrophilic functional group is N-hydroxy succinimide (NHS), which may be represented by the following Structural Formula 4:

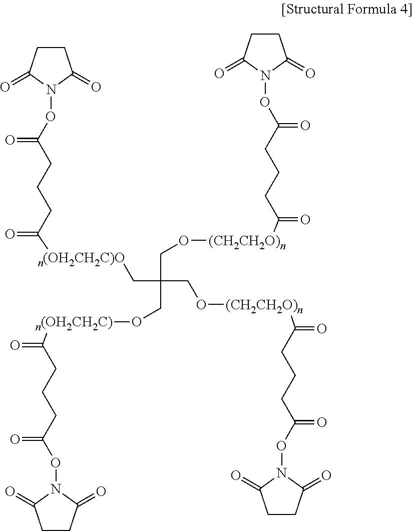

where in Structural Formula 4, n is an integer from 20 to 200.

The PEG derivative having a nucleophilic functional group may be a compound represented by the following Structural Formula 1B:

Core-[—(CH₂CH₂O)ₙ—(CH₂)ₘ₁-(L)ₚ-(CH₂)ₘ₂—R]_{q} [Structural Formula 1B]

where in Structural Formula 1B, L is a linker, may be each independently selected from the group consisting of

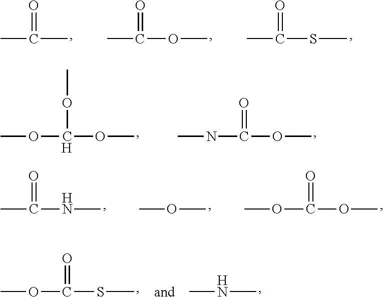

R is an NH₂ functional group,

Core may be selected from the group consisting of

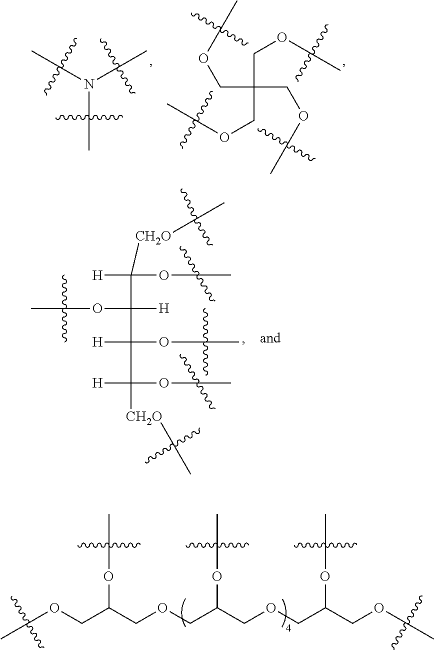

n is an integer from 10 to 2000,

m₁ and m₂ are each independent integers from 0 to 3,

p is an integer from 0 to 1,

q is an integer from 3 to 8.

An exemplary PEG derivative with nucleophilic functional group is a PEG derivative with an amine group (NH₂) and, more preferably, it is a compound represented by the following Structural Formula 6, but it not limited thereto:

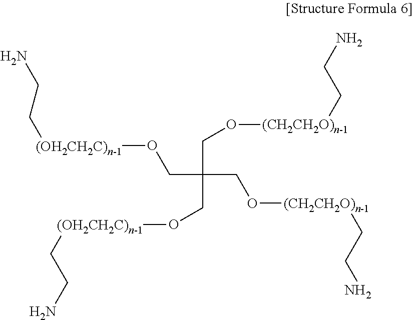

where in Structural Formula 6, n is an integer from 20 to 200.

Each of the PEG derivatives may be included at a concentration of 1 to 5% (w/v) in a phosphate buffer or physiological saline. If the concentration is lower than 1%, the composition has properties similar to those of a solution. If the concentration is higher than 5%, the composition has properties similar to those of hard gel, which results in higher viscoelasticity and makes it unsuitable as a biocompatible hydrogel. As the concentration of PEG derivatives increases and the reaction pH becomes more basic, the time required for hydrogel gelation decreases. It was confirmed that adding other ingredients, such as various pharmacological substances, to the PEG derivatives changes gelation time. It may be explained that adding other ingredients may shorten the physical distances between the PEG derivatives, which would facilitate gelation per unit hour.

Structural and physical properties of a hydrogel may be manipulated by its molecular weight, in addition to concentration and reaction conditions as previously mentioned. The larger the molecular weight becomes, the sparser the hydrogel structure becomes, and vice versa. In this embodiment, the PEG derivatives have molecular weight ranging from 1,000 to 100,000, and it is preferred that the molecular weight ranges from 5,000 to 20,000.

It is preferred to mix the PEG derivative with electrophilic functional group and the PEG derivative with nucleophilic functional group, in the molar ratio of 10:0.1˜10, 10:1˜10, 10:2˜9.5, 10:5˜9.5, or 10:6.5˜9.5.

Hyaluronic acid with a short half-life is added to the PEG hydrogel to form a hydrogel containing hyaluronic acid. The properties of the hydrogel may vary depending on the molecular weight or the concentration of the added hyaluronic acid. On the other hand, the half-life of the added hyaluronic acid may be influenced by the hydrogel. In the present invention, the added hyaluronic acid increases the elasticity of the hydrogel and preferably has the molecular weight range of 20,000 Da to 420,000 Da. The hyaluronic acid includes sodium hyaluronate.

In addition, the concentration of the hyaluronic acid may be between 0.05% (w/v) and 1% (w/v) due to variable viscosity depending on the molecular weights of the hyaluronic acid.

A hydrogel is defined as a composition that contains either natural or synthetic derivatives, which may swell without completely dissolving in aqueous solutions. In addition, a hydrogel has numerous advantages that may be applied in the biomedical field. In other words, a hydrogel displays much similarity to biological tissues as the hydrogel may absorb and retain aqueous solutions within the body, and it may also be permeable for low molecular weight substances, such as oxygen, nutrients and metabolites. Moreover, the surface of the swelled hydrogel is smooth, which would eliminate irritation caused by friction against surrounding cells or tissue within a body. Therefore, the present invention discloses a highly biocompatible and durable hydrogel as an injection for arthritis treatment by adding hyaluronic acid, which naturally has relatively short half-life, to a PEG-hydrogels, which is to be injected once into a joint (a synovial joint cavity) to cause an enhanced efficacy of pain relief, cartilage protection, and inhibition of synovial membrane inflammation without requiring surgery.

Also in the present invention, two different biocompatible polymers, in particular, PEG derivatives, may be reacted to form PEG-hydrogels by peptide bonds in either a neutral or a basic buffer.

In other words, when solution 1 and solution 2 are mixed, the PEG derivative with an electrophilic functional group and the PEG derivative with a nucleophilic functional group react to form peptide bond. More specifically, a PEG derivative with amine (NH₂) group and a NHS-containing chemical group form a peptide bond, as shown in the following Reaction 1:

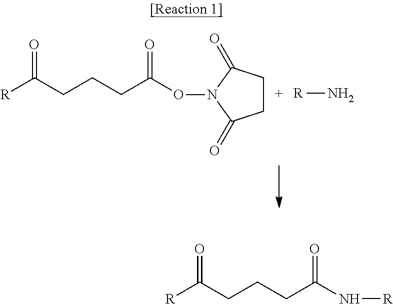

In the present invention, the rationale of setting different pHs for solution 1 and solution 2 is that the gelation occurs too rapidly if the pHs are identical in both solutions, in which case, the needle of the syringe becomes clogged. Therefore, the present invention exhibits a unique method, in which the two solutions have different pHs to manipulate the rate of gelation.

The injection of the present invention may be administered to a joint (a synovial joint cavity), and then a hydrogel forms after the injection. In the hydrogel, the values of elasticity and viscosity (G′, G″; Pa) change from low viscosity values close to those of a sol (0.3˜1 Pa) to high viscosity values close to those of a gel (≧300 Pa).

The complex viscosity of the hydrogel may have an initial value that ranges from 0.01 to 1 Pa·s, and it may range from 4 to 1,000 Pa·s at 2000 seconds or more.

In order to reduce the risk of causing pain due to high injection force during administration into a joint, which existing products have and results from the high viscosity of the products, the present invention provides a PEG-hydrogel containing hyaluronic acid that is easily injectable with a syringe due to low viscosity at the time of injection. Here, the low viscosity is achieved by controlling the duration of crosslinking, and the excellent viscoelasticity after injection is attained as a result of gradually reacting to form PEG-hydrogels by peptide bond.

As described above, the injection of the present invention displays high biocompatibility, easy injection, and biosustainability, thus a single injection of the present invention into a joint (a synovial joint cavity) may provide joint pain relief, protection of articular cartilages, and/or inhibition of synovial membrane inflammation. The overall volume of the injection ranges between 1 to 3 mL.

The injection of the present invention may also be provided as a kit.

In other words, the kit may contain two separate sets of solutions, wherein a buffer solution set 1 may contain a PEG derivative powder with an electrophilic functional group and a buffer solution of pH 3.5 to 6, which is stored separately from the powder; and a buffer solution set 2 may contain a PEG derivative powder with a nucleophilic functional group and a buffer solution of pH 7.5 to 11 containing hyaluronic acid, where the buffer solution is stored separately from the powder.

Each cylinder within a dual syringe may contain the solutions described above, and these solutions may be mixed just prior to injection to be administered as a single solution.

In the kit, the PEG derivative powder from the buffer solution 1 set is dissolved in solution 1 just before injection, and the PEG derivative powder from the buffer solution 2 set is dissolved in the hyaluronic-acid-containing buffer solution from the same set just before injection. Then, the solutions are mixed prior to injection.

Advantages and features of the present invention and methods of achieving the same will be clearly understood with reference to the following detailed embodiments. However, the present invention is not limited to the embodiments to be disclosed, but may be implemented in various different forms. The embodiments are provided in order to fully explain the present invention and fully explain the scope of the present invention for those skilled in the art. The scope of the present invention is defined by the appended claims.

Hereinafter, the present invention will be described in detail through examples. The following examples are merely provided to illustrate the present invention, and the scope of the present invention is not limited to the following examples. The examples are provided to complete the disclosure of the present invention and to fully disclose the scope of the present invention to those of ordinary skill in the art, and the present invention is only defined by the range of the appended claims.

### EXAMPLES

### Preparation Example 1. Synthesis of 4Arm PEG-Succinimidyl Glutarate (4Arm PEG-SG)

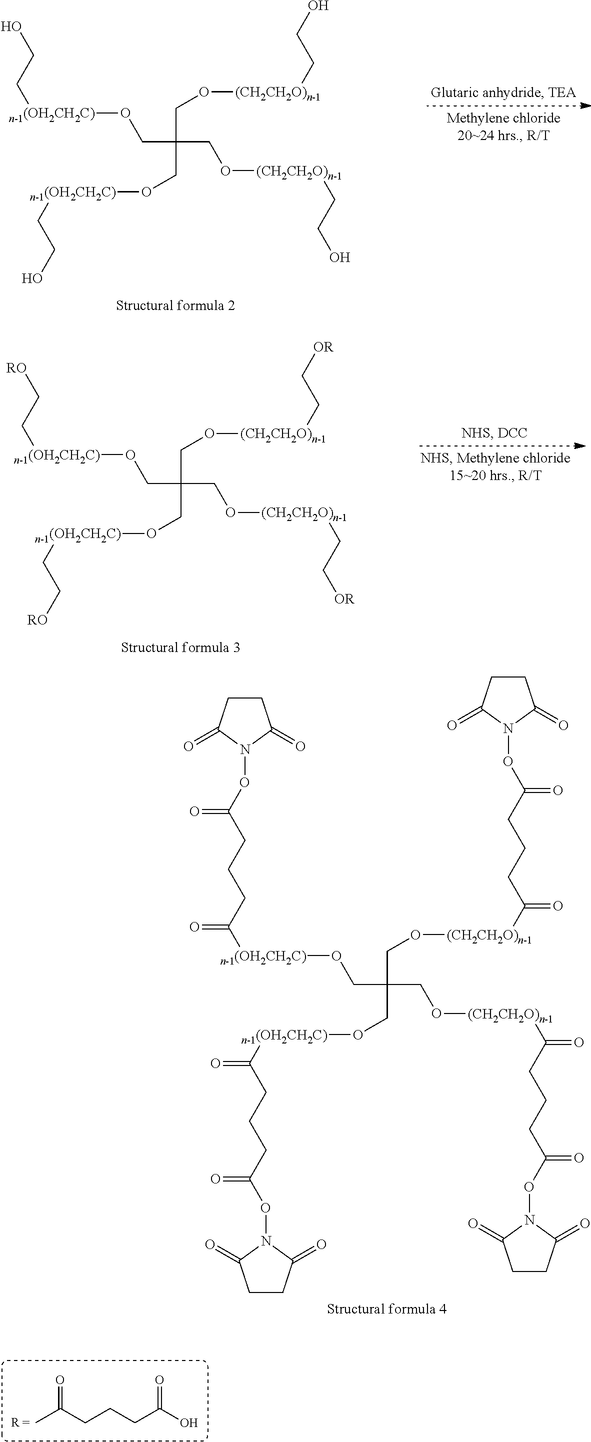

A compound of Structural Formula 2 was dissolved in methylene chloride at room temperature, and then triethylamine was added to the mixture. Glutaric acid anhydride (glutaric anhydride) was added to a reaction solution, and then stirred for 20 to 24 hours at room temperature. Then, the solution was washed with a 14% ammonium chloride solution. Once the liquid phases are separated, the organic phase in the bottom was collected. The aqueous phase was extracted by methylene chloride. The collected organic phase was treated with magnesium sulfate to remove moisture, and then precipitated by diethyl ether after concentrating the solvent. The precipitate was filtered and dried for 24 hours under vacuum at room temperature to yield a compound of Structural Formula 3.

The compound of Structural Formula 3 was dissolved in methylene chloride, and then N-hydroxysuccinimide (NHS) and dicyclohexyl carbodiimide (DCC) were added. The reaction solution was stirred for 15 to 20 hours at room temperature. Dicyclohexyl urea (DCU), which is a by-product of the reaction, was filtered using a glass filter, and the filtered solution was precipitated by diethyl ether after concentrating the solvent. Once the precipitate was filtered, the filtered precipitate was dissolved in ethyl acetate at 55±5° C. and re-crystallized for 15 to 17 hours at 0 to 5° C. The crystals were filtered, washed with diethyl ether 3 times, and vacuum-dried for 24 hours, which resulted in a compound (n=57) of Structural Formula 4 with average molecular weight of 10,000 Da.

### Preparation Example 2. Synthesis of 4Arm PEG-Amine

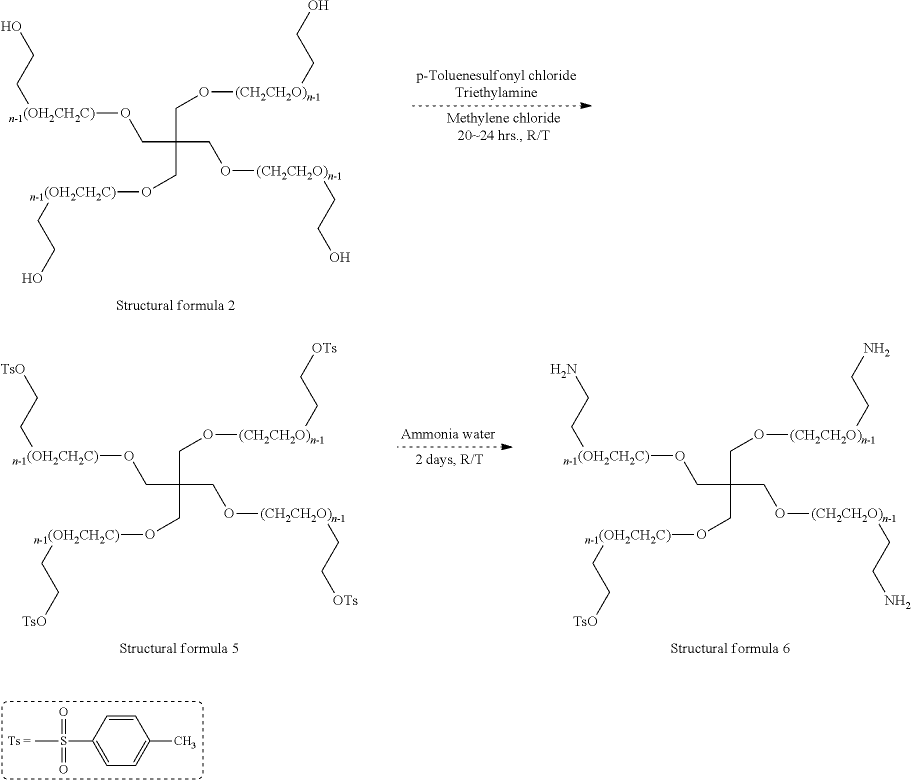

A compound of Structural Formula 2 was dissolved in methylene chloride at room temperature, and then triethylamine was added to the mixture. P-toluenesulfonyl chloride was added to the reaction solution, and then stirred for 20 to 24 hours at room temperature. Then, the solution was washed with a 14% ammonium chloride solution. Once the liquid phases are separated, the organic phase at the bottom was collected. The aqueous phase was extracted by methylene chloride. The collected organic phase was treated with magnesium sulfate to remove moisture, and then precipitated by diethyl ether after concentrating the solvent. The precipitate was filtered and dried for 24 hours under vacuum at room temperature to yield a compound of Structural Formula 5.

The compound of Structural Formula 5 was added to 28% ammonia, and stirred for 2 days at room temperature. Then, an organic phase was extracted twice after adding methylene chloride to the reaction solution. The collected organic phase was treated with magnesium sulfate to remove moisture, and then precipitated by diethyl ether after concentrating the solvent. The precipitate was filtered and dried for 24 hours under vacuum at room temperature to yield a compound (n=57) of Structural Formula 6 with average molecular weight of 10,000 Da.

### Examples 1˜3. Preparation of Hyaluronic Acid-PEG Hydrogel Injection

The PEG derivative (4arm-PEG-SG) prepared by the method described in Preparation Example 1 was dissolved in a phosphate buffered saline (PBS) buffer of pH 4.0 (Buffer A), which was sterilized at 121° C. for 15 minutes, in the amount according to the Table 1 to prepare Solution 1.

The PEG derivative (4arm-PEG-amine) prepared by the method described in Preparation Example 2 and hyaluronic acid (HA; High viscosity: 3.3, MW: 3,500,000˜4,200,000 Da; Bioland) were dissolved in a PBS buffer of pH 8.0 (Buffer B), which was sterilized at 121° C. for 15 minutes, in the amount according to the Table 1 to prepare Solution 2.

The two solutions were mixed in the volume ratio of 1:1 to form a hydrogel for injection into a joint (a synovial joint cavity). The viscosity of the hydrogel within 1 minute of mixing the two solutions is less than 0.5 Pa. The viscosity of 1% hyaluronic acid is generally 40 Pa.

TABLE 1

| | Solution 1 | Solution 2 |
|---|---|---|
| | 4arm-PEG-SG/ | 4arm-PEG-amine/ |
| | 5 ml Buffer A | 5 ml Buffer B |
| | (pH 4.0) | (pH 8.0) + 0.1% HA |
| Positive Control | — | — |
| Example 1 | 150 mg (0.003 mmole) | 105 mg (0.0021 mmole) |
| Example 2 | 150 mg (0.003 mmole) | 120 mg (0.0024 mmole) |
| Example 3 | 150 mg (0.003 mmole) | 135 mg (0.0027 mmole) |

As a positive control, 1% (10 mg/ml) hyaluronic acid (MW 3,500,000-4,200,000 Da; Bioland) was dissolved in PBS buffer pH 8.0, which was sterilized at 121° C. for 15 minutes.

### Test Example 1. Physical Properties of PEG Hydrogel Containing Hyaluronic Acid

Viscoelasticity of a PEG hydrogel containing hyaluronic acid was determined using a rheometer.

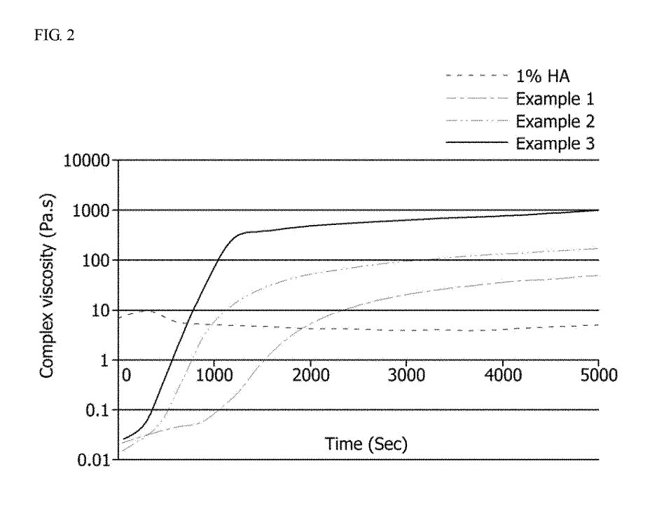

Using a DHR (Discovery Hybrid Rheometer, T.A Instruments, Ltd., USA) and a 40 mm plate, elasticity and viscosity values were determined with a 1% strain mode and an oscillation mode in constant 2.5 Hz at 37° C. The results are shown in FIG. 2.

As shown in FIG. 2, the complex viscosity of 1% hyaluronic acid (the positive control) did not change over time. However, the complex viscosity of the hyaluronic acid-PEG hydrogels of examples 1, 2, and 3 showed a gradual increase over time, and then plateaued after a certain period of time. In addition, as the mixture ratio of amine increases, the complex viscosity value increases. In other words, the viscoelasticity of the hydrogel is determined by the ratio of amine groups of PEG and PEG with NHS derivatives, followed by the degree of peptide bond formation. As the peptide bond ratio increases, the viscosity and the elasticity values increase. The resulting hydrogel is less deformed by an external force, and presents high durability and sustainability. The embodiment of the present invention is administering solutions after mixing two separate solutions, in which each contains PEG with either NHS or amine derivatives, in an 1:1 volume ratio. The formation of peptide bonds by mixing two different PEG derivatives initially generates a hydrogel with low viscoelasticity, which may lower the injection force to diminish pain of the patient during administration and make injection easier. Once administered into a synovial joint cavity, increased peptide bond formation results in a hydrogel with higher viscoelasticity, which extends the sustainability of the hydrogel.

### Test Example 2. Assessment of Analgesic Effect

Efficacy of the PEG hydrogel containing hyaluronic acid was investigated using the MIA-induced osteoarthritis rat model, which is commonly used to study osteoarthritis. Various compositions of hydrogel, examples 1, 2, and 3, were tested, and 1% hyaluronic acid was tested as a positive control.

Osteoarthritis was induced by MIA (monosodium iodoacetate, Sigma-Aldrich Co. LLC. Cat No. 19148) using a Hamilton syringe. 50 μl of MIA (60 mg/ml) was injected into a synovial joint cavity of a right knee of a rat after shaving the right knee and a surrounding region thereof (Corinne Guingamp et al., Mono-Iodoacetate-Induced Experimental Osteoarthritis, Arthritis & Rheumatism, 1997, 40(9), 1670-1679, Kai Gong et al., Journal of the Formosan Medical Association, 2011, 110(3), 145-152).

6 days after MIA injection, 50 μl of each of hydrogel composition of examples 1, 2, and 3, and the positive control, 1% hyaluronic acid (10 mg/ml), was injected into a synovial joint cavity.

The analgesic effects of the hydrogel were measured using an incapacitance tester (Stoelting Co., Wood Dale, Ill.) on days 4, 7, 14, and 28 after the MIA injection. The incapacitance tester measures the weight distribution on two hind paws; the force or the weight (g) exerted by each paw was measured. Based on the measured data, changes in hind paw weight distribution (HPWD, %) were calculated using the following Equation 1. The HPWD was measured three times for each rat.

% hind paw weight distribution=[left paw weight/(left paw weight+right paw weight)]×100 [Equation 1]

The measurements were calculated by the ratio of weight on a left paw with respect to weight on both paws, and expressed as mean (%)±standard deviation.

The ratio of changes in the weight on the left paw is the value obtained by calculating, in percentage, a ratio of additional weight exerted on the left hind paw due to the pain on the right knee as a result of induced arthritis on the right leg, and wildtypes without arthritis would display the ratio of 50%.

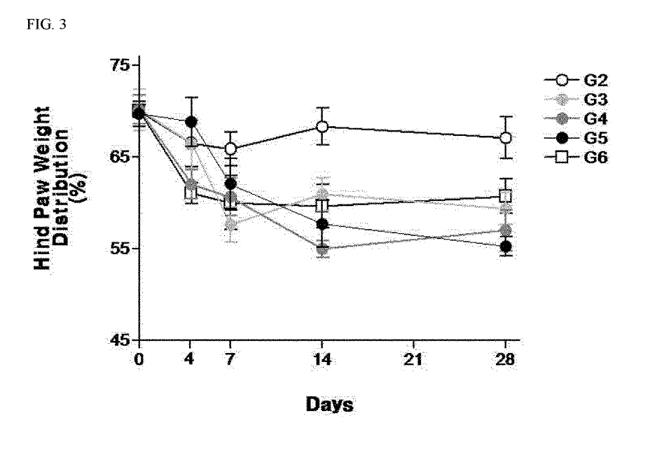

According to the experimental data, the ratio of hind paw weight distribution was at least 65% from day 4 to day 28 (when the rats were not treated with the hydrogel). On the other hand, the ratios of hind paw weight distribution decreased by 11.5%, 20.13%, and 16.19%, with respect to a vehicle control, on day 14 from the rats that were treated with the hydrogels of examples 1, 2, and 3, respectively. Hence, various compositions of the present invention showed significant therapeutic effects in all experimental groups. In addition, on day 28, the ratios of hind paw weight distributions reduced by 11.7%, 15.3%, and 17.8%, with respect to the vehicle control, in the groups that were treated with examples 1, 2, and 3, respectively, which showed significant therapeutic effects. In the positive control, the ratio of hind paw weight distribution reduced by 11.83% with respect to the vehicle control, which implies that the examples 1, 2, and 3 of the present invention have similar or even better analgesic efficacy than the positive control (Table 2 and FIG. 3).

TABLE 2

| GROUP | 4 days | 7 days | 14 days | 28 days |
|---|---|---|---|---|
| Wildtype (G1) | 49.65 ± 1.73** | 49.65 ± 2.03** | 49.57 ± 1.71** | 49.73 ± 0.95** |
| Vehicle Control (G2) | 64.67 ± 5.90 | 64.67 ± 4.29 | 68.86 ± 5.98 | 67.26 ± 6.85 |
| Example 1 (G3) | 66.45 ± 7.07 | 57.63 ± 5.15 | 60.94 ± 4.82 | 59.39 ± 4.62 |
| Example 2 (G4) | 62.02 ± 4.08 | 60.66 ± 5.44 | 55.00 ± 2.43 | 57.00 ± 5.95 |
| Example 3 (G5) | 68.82 ± 7.11 | 62.05 ± 7.44 | 57.71 ± 6.72 | 55.28 ± 2.87 |
| Positive control (G6) | 61.05 ± 3.27 | 60.02 ± 8.30 | 59.67 ± 6.70* | 60.72 ± 5.36* |

(Measurements were expressed as mean (%) ± standard deviation. The results were statistically analyzed by parametric One-Way ANOVA and Student' t-test methods (n = 7). *P < 0.05, **P < 0.01: with respect to the vehicle control (G2)).

### Test Example 3. Assessment of Histopathological Changes

The animals used in Test Example 2 were sacrificed using CO₂ gas. The right knee joint was separated from each animal, and was fixed with a 10% neutral formalin solution to perform Safranin-O staining to further assess histopathological changes.

The levels of joint damage in the rats treated with one of the vehicle control, the injections of examples 1, 2, and 3, and the positive control were examined and scored.

The scores were determined by assessing the degree of histopathological changes by osteoarthritis; presence of surface damage to the articular cartilage, amount of staining, changes in the number of cartilage cells and formation were included in the assessments (Mankin H J et al., J Bone Joint Surg Am. 1971 April; 53 (3):523-37).

TABLE 3

| | Group | | | | | | | | | | | |
|---|---|---|---|---|---|---|---|---|---|---|---|---|
| | | | Vehicle | | Example | | Example | | Example | | Positive | |
| | Wildtype | | control | | 1 | | 2 | | 3 | | control | |
| Categories₄³ | (G1) | | (G2) | | (G3) | | (G4) | | (G5) | | (G6) | |
| Structural changes in | 0.1 ± | 0.0** | 3.0 ± | 0.0 | 2.8 ± | 0.5 | 2.4 ± | 0.9 | 2.9 ± | 0.4 | 2.9 ± | 0.4 |
| the articular surface | | | | | | | | | | | | |
| irregularities | | | | | | | | | | | | |
| Fibrillation of | 0.0 ± | 0.0** | 3.0 ± | 0.0 | 2.6 ± | 0.5 | 1.3 ± | 1.3** | 2.8 ± | 0.5 | 2.6 ± | 0.5 |
| cartilage surface | | | | | | | | | | | | |
| Ulceration | 0.0 ± | 0.0** | 2.9 ± | 0.4 | 2.3 ± | 0.9 | 1.1 ± | 1.1** | 2.0 ± | 0.8* | 2.1 ± | 0.6* |
| Exposure of | 0.0 ± | 0.0** | 2.4 ± | 0.5 | 1.6 ± | 0.9 | 0.6 ± | 0.7** | 1.3 ± | 1.2* | 1.5 ± | 1.1 |
| subchondral bone | | | | | | | | | | | | |
| Degeneration/Necrosis | 0.0 ± | 0.0** | 2.9 ± | 0.4 | 2.3 ± | 0.5* | 2.0 ± | 0.5** | 2.1 ± | 0.6* | 2.5 ± | 0.5 |
| Replacement of | 0.0 ± | 0.0** | 2.4 ± | 0.7 | 1.8 ± | 0.7 | 1.1 ± | 0.6** | 1.5 ± | 0.8* | 1.5 ± | 0.8* |
| connective tissue | | | | | | | | | | | | |
| Increase in osteoclasts | 0.0 ± | 0.0** | 2.5 ± | 0.5 | 1.6 ± | 0.7* | 0.8 ± | 0.7** | 1.3 ± | 1.0* | 1.5 ± | 0.8* |
| Inflammatory cell | 0.0 ± | 0.0** | 2.5 ± | 0.8 | 1.5 ± | 0.5* | 0.8 ± | 0.5** | 1.1 ± | 0.4* | 1.3 ± | 0.9* |
| infiltration in synovial | | | | | | | | | | | | |
| tissue | | | | | | | | | | | | |
| Synovial cell | 0.1 ± | 0.4** | 2.6 ± | 0.5 | 2.0 ± | 0.5* | 1.9 ± | 0.4** | 2.0 ± | 0.5* | 1.9 ± | 0.6** |
| proliferation | | | | | | | | | | | | |
| Reduction of | 0.0 ± | 0.0** | 2.6 ± | 0.5 | 2.4 ± | 0.5 | 2.4 ± | 0.5 | 2.3 ± | 0.7 | 2.5 ± | 0.5 |
| Safranin-O staining in | | | | | | | | | | | | |
| cartilage | | | | | | | | | | | | |

(Measurement were expressed as mean (%) ± standard deviation. The results were statistically analyzed by non-parametric Kruskal-Wallis and Mann-Whitney testing. Moderate changes were marked as 1, intermediate changes were marked as 2, and severe changes were marked as 3. *P < 0.05, **P < 0.01: with respect to the vehicle control (G2))

Once the histopathological assessment was conducted, findings from each group were compared for each category of the assessments. In wildtype (G1), there were no osteoarthritis-related findings except few negligible changes in some animals. On the other hand, the vehicle control (G2), in which osteoarthritis was induced with MIA, displayed the most severe formation of lesions among all groups of rats.

Among the categories of histopathological assessments, the measurements of the structural changes and surface irregularities in the joints indicated that the experimental groups administered with examples 1, 2, and 3 (G3, G4, G5), and the positive control (G6) did not show statistically significant differences compared to the vehicle control (G2).

When the levels of cartilage surface fibrillation were measured, the group administered with example 2 (G4) showed a statistically significant decrease in the level of fibrillation compared to the vehicle control (G2) (p<0.01). Interestingly, the positive control (G6) did not show any statistically significant difference compared to the vehicle control (G2).

When the levels of ulceration were measured, the groups administered with examples 2 and 3 (G4, G5) showed a statistically significant decrease compared to the vehicle control (G2) (p<0.05 or p<0.01). The positive control (G6) also showed a statistically significant decrease compared to the vehicle control (G2) (p<0.05).

When the levels of subchondral bone exposure were measured, the groups administered with examples 2 and 3 showed a statistically significant decrease compared to the vehicle control (G2) (p<0.05 or p<0.01). Interestingly, the positive control (G6) did not show any statistically significant difference compared to the vehicle control (G2).

When the levels of chondrocytes degeneration/necrosis were measured, the experimental groups administered with examples 1, 2, and 3 (G3, G4, G5) showed a statistically significant decrease compared to the vehicle control (G2) (p<0.05 or p<0.01). Interestingly, the positive control (G6) did not show any statistically significant difference compared to the vehicle control (G2).

When the levels of replacement of fibrous tissue were measured, the groups of examples 2 and 3 showed a statistically significant decrease compared to the vehicle control (G2) (p<0.05 or p<0.01). The positive group (G6) also showed a statistically significant decrease compared to the vehicle control (G2) (p<0.05).

When the levels of an increase in osteoclasts were measured, the groups administered with examples 1, 2 and 3 (G3, G4, G5) showed a statistically significant decrease compared to the vehicle control (G2) (p<0.05 or p<0.01). The positive group (G6) also showed a statistically significant decrease compared to the vehicle group (G2) (p<0.05).

When the levels of inflammatory cell infiltration in synovial tissue were measured, the groups administered with examples 1, 2 and 3 (G3, G4, G5) showed a statistically significant decrease compared to the vehicle control group (G2) (p<0.05 or p<0.01). The positive group (G6) also showed a statistically significant decrease compared to the vehicle group (G2) (p<0.05).

When the levels of synovial cell proliferation in synovial membrane were measured, the groups administered with examples 1, 2 and 3 (G3, G4, G5) showed a statistically significant decrease compared to the vehicle control group (G2) (p<0.05 or p<0.01). The positive group (G6) also showed a statistically significant decrease compared to the vehicle group (G2) (p<0.05).

When the levels of reduction of staining in cartilage were measured through Safranin-O staining, there was no statistically significant decrease in the groups administered with chemicals and in the positive group compared to the vehicle control group (G2).

The group administered with example 1 (G3) measured higher in most of the categories compared to the groups administered with example 2 (G4) or 3 (G5). The positive control group (G6) measured lower compared to the group administered with example 1 (G3) in most categories except for the categories of degeneration/necrosis and the amount of Safranin-O staining. The groups administered with example 2 (G4) or 3 (G5) showed the largest overall improvement compared to the vehicle control group (G2). The group administered with example 2 (G4) had a lower average in the categories except for the amount of Safranin-O staining in cartilages compared to the group administered with example 3 (G5). Although the group administered with example 3 (G5) was found to have a higher average of measurements in most categories compared to the group administered with example 2 (G4), there was no significant difference among groups in some categories such as degeneration/necrosis and lesions in synovial membranes.

### Test Example 4. Intra-Articular Clearance of Radioactivity in Rabbit Knees Following a Single Intra-Articular Dose

“Intra-articular clearance of radioactivity in rabbit knees following a single intra-articular dose of PEG hydrogel containing HA”, conducted by MPI Research, Inc. The [¹⁴C]4arm PEG-AM, PEG derivative (4arm-PEG-AM) labeled with ¹⁴C, prepared from Curachem (Korea) by the method described in Preparation Example 2. The objective of this study was to determine the rate of intra-articular clearance of radioactivity in rabbit knee joints, following a single intra-articular injection of [¹⁴C] example 2 in the Table 1.

One treatment group of seven male New Zealand White Hra:(NZW)SPF albino rabbits was administered the [¹⁴C] example 2 once via intra-articular injection into both right and left knees.

One animal was euthanized at 1 hour postdose to establish baseline control levels. Additional animals were euthanized at 1, 2, 4, 6, 8 and 10 weeks postdose, and the knees removed and submitted for radioanalysis by LSC (Liquid Scintillation Counting). The knees were severed approximately one-half inch above and below the joint. Left and right knees were processed separately. The intact joint was first solubilized in strong base solution and placed in an oven at 60° C. overnight. The next day, the remaining bone was removed from the solubilized soft tissue, and placed in strong aid solution and placed in an oven at 60° C. overnight. Both the soft tissue and bone were analyzed by LSC, and the counts for each summed to give total counts. It should be noted that the majority of counts were noted in the soft tissue, and assumed to be within the articular space. There were very few counts noted in the bone for each animal.

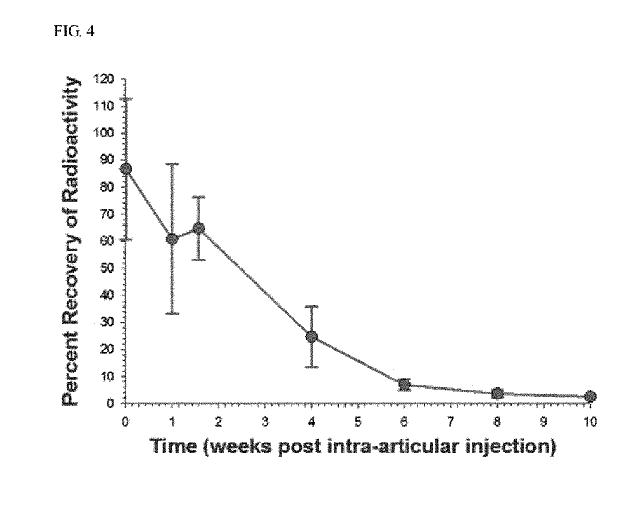

The following graph (FIG. 4) and summary table (Table 4) demonstrate the radioactivity recovered following dosing from the knees, over time. The data for the left and right knees for each time point shown were averaged. It should be mentioned that even though both knees were averaged, the results represent only one animal per time point, and there was high variability observed at the earlier time points. At 1 hour postdose, the established baseline, the radioactivity recovered ranged from 68.30 to 105.09%, with an average of 86.70%. At 1 week postdose, the radioactivity recovered ranged from 41.19 to 80.34%, with an average of 60.77%. At 11 days postdose (animal died prematurely), the radioactivity recovered ranged from 56.61 to 72.90%, with an average of 64.76%. At 4 weeks postdose, the radioactivity recovered ranged from 16.84 to 32.62%, with an average of 24.73%. All values beyond 4 weeks postdose (6, 8, and 10 weeks postdose) had recovery values less than 10%, and variability between the left and right knees was observed to be much less. While pharmacokinetic statistics were not used to determine the exact half-life of the [¹⁴C] example 2, a rough estimate would be between two and four weeks.

TABLE 4
Summary of Rabbit Knee Percent (%) Recovery Values

| | | | | Standard |
|---|---|---|---|---|
| Time Point | Right | Left | Average | Deviation |
| 1 hour | 68.30 | 105.09 | 86.70 | 26.01 |
| 1 week | 80.34 | 41.19 | 60.77 | 27.68 |
| 11 days* | 56.61 | 72.90 | 64.76 | 11.52 |
| 4 weeks | 16.84 | 32.62 | 24.73 | 11.16 |
| 6 weeks | 5.57 | 8.35 | 6.96 | 1.97 |
| 8 weeks | 4.64 | 2.67 | 3.66 | 1.39 |
| 10 weeks | 2.10 | 2.98 | 2.54 | 0.62 |

*Animal euthanized early (11 days)

Native hyaluronic acid has a relatively short half-life (shown in rabbits) so various manufacturing techniques have been deployed to extend the length of the chain and stabilize the molecule for its use in medical applications. Usually, most viscosupplements contain 5-15 mg/ml HA and, once injected, have residence half-life between hours to several days.

Products in the form of crosslinked hyaluronic acid, such as Synvisc® was studied intra-articular clearance in rabbit and disclosed result to Synvise-“summary of safety and effectiveness data (Non-Patented Reference 3)”. As Shown in Table 5, the clearance was measured using Synvisc® made from a combination of radiolabeled hylan A and hylan B.

TABLE 5
Intra-Articular Clearance Studies in Rabbits

| Test Article | | Half-Life |
|---|---|---|
| [³H]-hylan A fluid (1%)(avg. MW; 6 million) | | 1.2 ± 0.1 days |
| [³H]-hylan B gel (0.4%) | | 7.7 ± 1.0 days |
| [³H]/[¹⁴C]- Synvisc ® | ¹⁴C-hylan A fluid | 1.5 ± 0.2 days |
| | ³H-hylan B gel | 8.8 ± 0.9 days |

| | |
|---|---|
| Hyaluronan 1% (avg. M.W.: 1.7~2.6 million) | 11 hours |

Hylan A and Hylan B had a half-life in intra-articular of 1.2 ± 0.1 days, 7.7 ± 1.0 days. The longer half-life of hylan A may reflect the higher molecular weight of the cross-linked hylan as compared to native hyaluronan(11 hours). Synvisc ®, a combination of hylan A and hylan B, had a half-life in intra-articular of 8.8 ± 0.9 days.

In conclusion, it can be seen that the half-life of Example 2 (between two and four weeks) is two times longer than that of Synvisc® (8.8±0.9 days).

What is claimed is:

1. An injection composition comprising two separate solutions, the injection composition comprising:
a first solution comprising a first polyethylene glycol derivative having an electrophilic functional group, and a buffer solution having a pH of 3.5 to 6; and
a second solution comprising hyaluronic acid, a second polyethylene glycol derivative having a nucleophilic functional group, and a buffer solution having a pH of 7.5 to 11.

2. The injection composition of claim 1, wherein the first solution and the second solution are mixed just prior to injection.

3. The injection composition of claim 1, wherein the injection composition forms a hydrogel after injection.

4. The injection composition of claim 1, wherein the first polyethylene glycol derivative is represented by Structural Formula 1A as follows:
Core-[—(CH₂CH₂O)ₙ—(CH₂)ₘ₁-(L)ₚ-(CH₂)ₘ₂—R]_{q}
wherein L is a linker, and each L is independently selected from the group consisting of 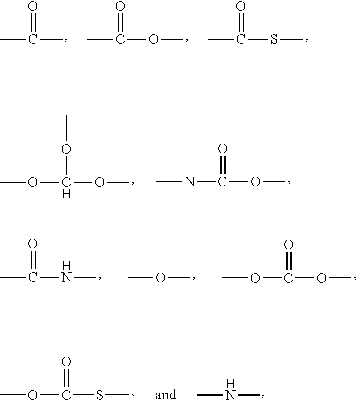
R is selected from the group consisting of 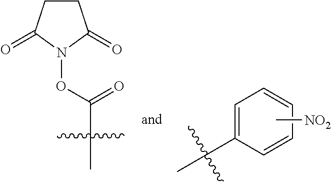
wherein R reacts with an amine group to form a peptide bond,
Core is selected from the group consisting of 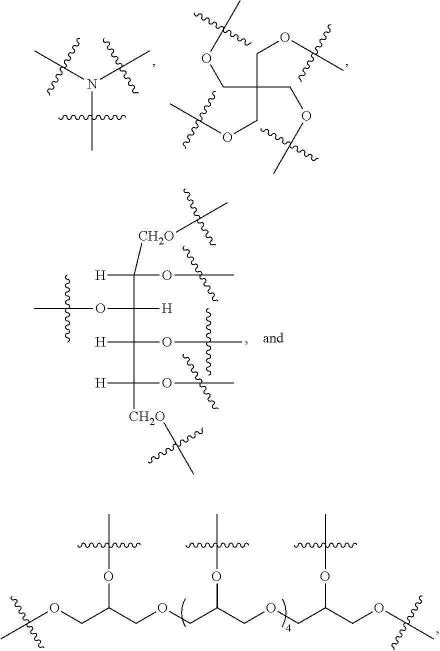
n is an integer from 10 to 2000,
m₁ and m₂ are each independently integers from 0 to 3,
p is 0 or 1, and
q is an integer from 3 to 8.

5. The injection composition of claim 1, wherein the second polyethylene glycol derivative is represented by Structural Formula 1B as follows:
Core-[—(CH₂CH₂O)ₙ—(CH₂)ₘ₁-(L)ₚ-(CH₂)ₘ₂—R]_{q} (1B)
wherein, L is a linker, and each L is independently selected from the group consisting of 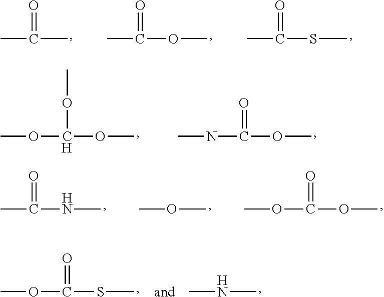
R is a NH₂ functional group,
Core is selected from the group consisting of 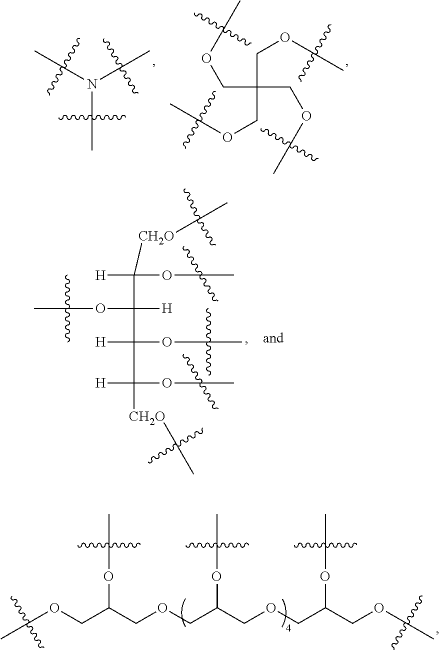
n is an integer from 10 to 2000,
m₁ and m₂ are each independently integers from 0 to 3,
p is 0 or 1, and
q is an integer from 3 to 8.

6. The injection composition of claim 1, wherein the first polyethylene glycol derivative is represented by Structural Formula 4 as follows: 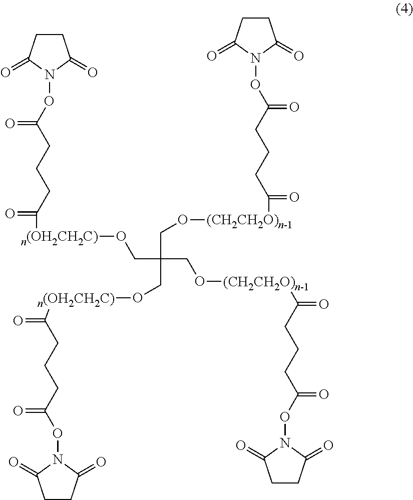
wherein, n is an integer from 20 to 200.

7. The injection composition of claim 1, wherein the second polyethylene glycol derivative is represented by Structural Formula 6 as follows: 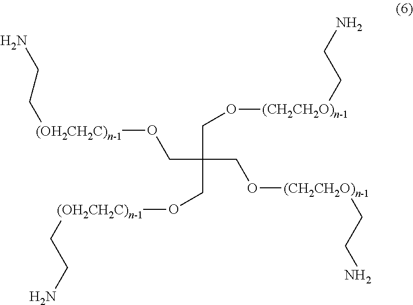
wherein n is an integer from 20 to 200.

8. The injection composition of claim 1, wherein the hyaluronic acid has a molecular weight ranging from 20,000 Da to 4,200,000 Da.

9. The injection composition of claim 1, wherein the hyaluronic acid is included at a concentration ranging from 0.05% (w/v) to 1% (w/v).

10. The injection composition of claim 1 wherein, upon mixing of the first solution and the second solution, a peptide bond is formed between the first polyethylene glycol derivative and the second polyethylene glycol derivative.

11. The injection composition of claim 2, wherein the first polyethylene glycol derivative and the second polyethylene glycol derivative are mixed at a molar ratio of between 10:0.1 and about 10:10.

12. The injection composition of claim 3, wherein the hydrogel initially has a complex viscosity that ranges from 0.01 Pa·s to 1 Pa·s.

13. The method according to claim 14, wherein the injection composition is administered into a joint cavity.

14. A method of relieving pain, protecting cartilage, or inhibiting synovial membrane inflammation, the method comprising administering the injection composition of claim 1 to a subject in need thereof.

15. A kit for an injection comprising two separate sets of solutions, the kit comprising:
a first buffer solution set comprising a first polyethylene glycol derivative powder having an electrophilic functional group, and a first buffer solution having a pH of 3.5 to 6, wherein the first buffer solution is stored separately from the first polyethylene glycol derivative powder; and
a second buffer solution set comprising a second polyethylene glycol derivative powder having a nucleophilic functional group and a second buffer solution having a pH of 7.5 to 11 containing hyaluronic acid, wherein the second buffer solution is stored separately from the second polyethylene glycol derivative powder.

16. A method of preparing the kit of claim 15 for injection, wherein (i) the first polyethylene glycol derivative powder from the first buffer solution set is dissolved in the first buffer solution just prior to injection to prepare a first solution, (ii) the second polyethylene glycol derivative powder from the second buffer solution set is dissolved in the second buffer solution containing hyaluronic acid just prior to injection to prepare a second solution, and (iii) the first solution and the second solution are mixed just prior to injection.
